(19) United States
(12) Patent Application Publication
(10) Pub. No.: US 2025/0129831 A1
(43) Pub. Date: Apr. 24, 2025

(54) Spring Carrier

(71) Applicant: Sanofi Winthrop Industrie, Gentilly (FR)

(72) Inventors: Philippe Chabert, Paris (FR); David Darras, Paris (FR)

(21) Appl. No.: 18/572,445

(22) PCT Filed: Jun. 23, 2022
(86) PCT No.: PCT/EP2022/067087

§ 371 Date: Dec. 20, 2023

(30) Foreign Application Priority Data
Jun. 24, 2021 (EP) 21315105.3

(51) Int. Cl. F16F 1/12; A61M 5/32; B23P 19/04

(52) U.S. Cl. CPC F16F 1/12; A61M 5/321; B23P 19/048; A61M 2207/10; F16F 2226/04; F16F 2230/10

(57) ABSTRACT

A spring carrier for receiving, retaining, and discharging of a coil spring in a manufacturing assembly process includes an elongate hollow body defining an inner cavity to receive the coil spring and an opening at a proximal first end of the hollow body for the insertion of the coil spring into the inner cavity and extraction of the coil spring from the inner cavity. A deflectable member is located proximate to the first proximal end and includes a retaining portion that retains the coil spring when the coil spring is located within the inner cavity. The deflectable member is movable between a first unbiased position, where the retaining portion extends into the inner cavity to retain the coil spring within the inner cavity, and a second biased position, where the retaining portion is disposed outwardly to allow the coil spring to be extracted from the inner cavity through the opening.

### CROSS REFERENCE TO RELATED APPLICATIONS

The present application is the national stage entry of International Patent Application No. PCT/EP2022/067087, filed on Jun. 23, 2022, and claims priority to Application No. EP 21315105.3, filed on Jun. 24, 2021, the disclosures of which are incorporated herein by reference.

### TECHNICAL FIELD

The present disclosure relates to a device for carrying a spring, an apparatus including such a device, and a method of use of such a device and apparatus.

### BACKGROUND

Many devices require one or more springs and the method and apparatus for assembly of such devices requires accurate and repeated retrieval, movement and placement of such springs. Devices including one or more springs in their assembly include medicament injection devices. Such devices may include a spring to facilitate various functions of the device, including operation of a drug administration mechanism, or deployment of one or more safety features before, during or after a medicament delivery process.

Springs can readily become entangled if stored or conveyed together in a bulk manner, and separating springs when required to be assembled into a device being manufactured can be difficult and time consuming, and therefore inefficient and costly in terms of the manufacturing process. In high-volume manufacturing processes, errors in an assembly line or a need to pause a production line, for example due to a jam or fault in the machinery, is undesirable as it leads to lost production time, loss of productivity and product output, and impacts manufacturing and product costs.

In a manufacturing process of a product containing one or more springs, it is therefore desirable to provide a device that facilitates repeated and reliable retrieval, transport, and placement of such springs for use in such a process, and/or which may help protect and ensure spring integrity.

### SUMMARY

According to the present disclosure, there is provided a spring carrier for receiving, retaining and discharging of a coil spring in a manufacturing assembly process comprising an elongate hollow body defining an inner cavity configured to receive a coil spring, an opening at a first proximal end of the hollow body for the insertion and extraction of a coil spring into and from the inner cavity, the hollow body including a second distal end opposite to the first proximal end, at least one deflectable member located proximate to the first proximal end of the hollow body and including a retaining portion which is configured to retain a coil spring when located within the inner cavity, wherein the deflectable member is movable between first unbiased position, whereby the retaining portion extends into the inner cavity to retain the coil spring within the inner cavity, and a second biased position, whereby the retaining portion is disposed outwardly to allow the coil spring to be extracted from the inner cavity through the opening.

The retaining portion may extend further outwardly when the deflectable member is in the second, biased position than when the deflectable member is in the first, unbiased position. The retaining portion being disposed outwardly in the second biased position may comprise being outwardly relative to a central axis or a surface of a side wall of the spring carrier, and may be radially outwardly thereof. The retaining portion may engage a coil spring when located within the inner cavity. Such engagement may comprise contact with the coil spring, and/or blocking of the coil spring, and/or restriction of movement of the coil spring within the inner cavity, and/or prevention of the coil spring from being removed from the inner cavity.

The retaining portion may be disposed outwardly of an inner surface of the inner cavity when the deflectable member is in the second biased position.

The deflectable member may be in a relaxed state in the first unbiased position and may be elastically deformed in the second biased position.

The deflectable member may extend substantially parallel to a central axis of the hollow body in the first unbiased position. The or each deflectable member may extend in a substantially longitudinal direction of the elongate hollow body.

The deflectable member may include an actuation feature for engagement with an actuator to deflect the deflectable member from the first unbiased position to the second biased position.

The actuation feature may comprise a contact surface disposed at an acute angle with respect to the central axis of the hollow body when the deflectable member is in the first unbiased position.

The contact surface may be located on a proximal end of the deflectable member, and the proximal end may be disposed further towards the first proximal end of the hollow body than the remainder of the deflectable member.

The contact surface may be disposed further in a proximal direction than the remainder of the hollow body when the deflectable member is in the first unbiased position.

The contact surface may be disposed substantially perpendicular with respect to the central axis of the hollow body when the deflectable member is in the second biased position.

The retaining portion may comprise at least one projecting region extending inwardly from the deflectable member.

The projecting region may be disposed proximate to the proximal end of the deflectable member.

The retaining portion may further comprise at least one projecting element extending inwardly from the deflectable member.

The or each projecting element may be provided on a plurality of deflectable members. The or each projecting element on one deflectable member may be aligned in an axial direction of the hollow body with the or each corresponding projecting element on another deflectable member. The or each projecting element on one deflectable member may be off-set in an axial direction of the hollow body with the or each corresponding projecting element on another deflectable member.

The projecting elements provided on the or each deflectable member may be of different sizes to each project by a different distance from the or each deflectable member. The projecting elements may increase in size and/or projecting distance in a direction towards a free end of the or each deflectable member, and/or in a direction towards the first proximal end of the hollow body.

The retaining portion may be disposed inwardly of an axial projection of an internal surface of the inner cavity in the first unbiased position, and may be disposed at least level or outwardly of an axial projection of the internal surface of the inner cavity in the second biased position.

The hollow body may include a flange proximate the first proximal end of the hollow body and extending radially outwardly from the hollow body. The flange may extend around the perimeter of the hollow body and be interrupted by the at least one deflectable member.

The flange may include an abutment surface facing in a direction of the first end of the hollow body.

The contact surface may extend in the same plane as the abutment surface when the deflectable member is in the second biased position.

The contact surface may extend in a plane oriented at an acute angle relative to a plane of the abutment surface when the deflectable member is in the first unbiased position.

The contact surface may extend further in an axial direction of the first end of the hollow body than the abutment surface when the deflectable member is in the first unbiased position.

The deflectable member may be integrally formed with a side wall of the hollow body.

The deflectable member may be disposed within an aperture in a side wall of the hollow body.

The deflectable member may comprise a resilient arm.

The spring carrier may comprise a plurality of deflectable members. The plurality of deflectable members may be equally spaced around the perimeter of the hollow body. The spring carrier may comprise two deflectable members disposed diametrically opposite to each other on the hollow body.

The hollow body may comprise a cylindrical tube which is circular in cross-section. The hollow body may be substantially uniform in cross-section dimension along its length. The hollow body may vary in cross-sectional dimension along its length.

The hollow body may be substantially rigid and not readily deformable from its cross-sectional shape. The or each deflectable member may be deflectable between the first and second positions relative to a side wall of the hollow body.

The spring carrier may comprise an opening at the second distal end of the hollow body. The opening at the second distal end of the hollow body may be of the same cross-sectional dimension as the cross-sectional dimension of the inner cavity.

The opening at the second distal end of the hollow body may be of a smaller cross-sectional dimension than the cross-sectional dimension of the inner cavity.

The second distal end of the hollow body may be closed.

The spring carrier may comprise at least one window to allow a coil spring located within the spring carrier to be visible from outside the spring carrier through the window. The or each window may be formed in a side wall of the hollow body, and may be formed in a side wall of the hollow body in a location between the first proximal end and the second distal end of the hollow body. The or each window may be formed in the at least one deflectable member. The or each window may be formed in one or both of the side wall and the or each delectable member.

The second distal end of the hollow body may include one or more protrusions extending inwardly from the hollow body. The or each protrusion may extend at least partially across an opening at the second distal end of the hollow body. The second end of the hollow body may include an inwardly-protruding wall or lip extending at least partially around an opening at the second distal end. The second distal end of the hollow body may be partially or entirely closed by an end wall.

The spring carrier may comprise one or more orientation features configured for cooperation with corresponding orientation features on an apparatus with which the spring carrier may be used. The orientation feature(s) may allow the spring carrier to be accurately aligned in use. Such orientation feature(s) may comprise one or more recesses or slots in the flange. Such orientation feature(s) may comprise diametrically opposed slots in the flange.

The or each deflectable member may be configured to deflect laterally outwardly in the second position by a distance of 1 mm-5 mm, and may be between 1 mm-3 mm, and may be between 1-2 mm, and may be around 1.5 mm.

The or each deflectable member may be configured to deflect laterally outwardly in the second position by an angle of around 4 to 20 degrees, and may be between 5 to 15 degrees, and may be around 10 degrees.

The spring carrier may comprise one or more centering lugs projecting inwardly from an inside surface of a side wall of the hollow body. The centering lugs may project towards the central axis of the hollow body. The centering lugs may be equally spaced around the inside circumference of the side wall of the hollow body. The or each centering lug may be formed as ramp which increases in inward projecting distance in a direction towards the second distal end of the hollow body.

Also provided in the present disclosure is an apparatus comprising a spring carrier as described above, and an actuator configured for engagement with the deflectable member and operable to move the deflectable member from the first unbiased position to the second biased position.

The actuator may comprise a planar surface and the deflectable member may comprise a free end proximate the first proximal end of the spring carrier configured such that pressing the first proximal end of the spring carrier onto the planar surface causes the deflectable member to be moved from the first unbiased position to the second biased position.

The contact surface may be configured to engage the planar surface when the first proximal end of the spring carrier is pressed against the planar surface.

The abutment surface may lie in contact with the planar surface when the first proximal end of the spring carrier is pressed against the planar surface and the deflectable member is in the second biased position.

The actuator may comprise an actuation plate. The actuator may comprise two or more actuation plates spaced apart. A coil spring may be inserted into and extracted from the inner cavity through a gap between the spaced apart actuation plates. An actuation plate may include a feed aperture therethrough for alignment with the inner cavity of the hollow body such that a coil spring can be inserted into and extracted from the inner cavity through the feed aperture when the spring carrier is engaged with the actuator.

The feed aperture may comprise a first region of substantially uniform cross-sectional dimension. The cross-sectional dimension of the first region may be substantially the same as the cross-sectional dimension of the inner cavity.

The feed aperture may comprise a second region comprising an outwardly tapering region extending between the first region and the planar surface such that the cross-sectional area of the feed aperture at the planar surface is greater than the cross-sectional area of the feed aperture at the first region.

The feed aperture may comprise a third region comprising an outwardly tapering region extending between the first region and a second planar surface of the actuation plate on the opposite side thereof to the first planar surface such that the cross-sectional area of the feed aperture at the second planar surface is greater than the cross-sectional area of the feed aperture at the first region.

The feed aperture may comprise one or both of the second and third outwardly-tapering regions.

An inner face of the or each deflectable member at a point of contact with the planar surface may be disposed radially outwardly from the central axis of the hollow body at least as far as the edge of the feed aperture at the planar surface.

The apparatus may further comprise an airflow generator configured to generate a flow of air into the hollow body to facilitate extraction of a coil spring from the hollow body.

The spring carrier may include an airflow passage at the second end of the hollow body to allow the flow of air into and through the hollow body from the airflow generator.

The airflow generator and/or the airflow passage may be configured to direct the flow of air into the hollow body at an acute angle other than parallel relative to the central axis of the hollow body.

The airflow generator may comprise an air duct connectable or insertable to/into the second end of the hollow body.

Also provided in the present disclosure is a manufacturing apparatus comprising an apparatus described above, and a spring extraction station configured to receive the spring carrier and locate the spring carrier whilst the actuator is engaged with the spring carrier to allow extraction of the coil spring from the spring carrier.

Also provided in the present disclosure is an assembly system comprising an apparatus as described above, and a coil spring manufacturing machine, wherein the coil spring manufacturing machine is configured to produce a coil spring, and the system further includes an insertion station arranged to feed the produced coil spring into the spring carrier.

The assembly system may further include the manufacturing apparatus comprising the extraction station described above.

Also provided in the present disclosure is a method of manipulating a coil spring using a spring carrier as described above, the method comprising moving the deflectable member from the first position to the second position, inserting the coil spring into the inner cavity through the opening at the first end of the hollow body, and moving the deflectable member from the second position to the first position such that the retaining portion extends into the inner cavity to retain the coil spring within the inner cavity.

Also provided in the present disclosure is a method of manipulating a coil spring using a spring carrier as described above, the method comprising moving the deflectable member from the first position to the second position such that the retaining portion does not retain the coil spring within the inner cavity to allow the coil spring to be extracted from the inner cavity through the opening at the first proximal end of the hollow body.

Also provided in the present disclosure is a method of manipulating a coil spring using a spring carrier for receiving, retaining and discharging of a coil spring in a manufacturing assembly process, the spring carrier comprising an elongate hollow body defining an inner cavity, an opening at a proximal first end of the hollow body, a second distal end opposite to the first proximal end, and at least one deflectable member located proximate to the first proximal end of the hollow body and including a retaining portion, the method comprising moving the deflectable member from a first position whereby the retaining portion extends into the inner cavity, to a second position whereby the retaining portion is disposed outwardly, inserting the coil spring into the inner cavity through the opening at the first end of the hollow body, and moving the deflectable member from the second position to the first position such that the retaining portion extends into the inner cavity to retain the coil spring within the inner cavity.

Also provided in the present disclosure is a method of manipulating a coil spring using a spring carrier for receiving, retaining and discharging of a coil spring in a manufacturing assembly process, the spring carrier comprising an elongate hollow body defining an inner cavity, an opening at a proximal first end of the hollow body, a second distal end opposite to the first proximal end, and at least one deflectable member located proximate to the first proximal end of the hollow body and including a retaining portion, the method comprising moving the deflectable member from a first position whereby the retaining portion extends into the inner cavity, to a second position whereby the retaining portion is disposed outwardly such that the retaining portion does not retain the coil spring within the inner cavity to allow the coil spring to be extracted from the inner cavity through the opening at the first proximal end of the hollow body.

The method may comprise the retaining portion not extending into the inner cavity in the second position of the deflectable member.

The method may comprise engaging an actuator with the deflectable member to move the deflectable member from the first position to the second position, and disengaging the actuator after insertion of the coil spring into the inner cavity to allow the deflectable member to move to the first position such that the retaining portion retains the coil spring within the inner cavity.

The step of engaging the actuator with the deflectable member may comprise pressing the first proximal end of the spring carrier against a planar surface of the actuator such that the deflectable member contacts the planar surface and is moved to the second position.

The first proximal end of the spring carrier may be pressed against the planar surface until the flange contacts the planar surface, at which point the deflectable member may be in the second position.

The actuator may comprise an actuation plate, and the step of inserting the coil spring into and/or extracting the coil spring from the inner cavity may comprise passing the coil spring through a feed aperture in the actuation plate and into/out of the inner cavity . . .

The spring carrier may comprise a window in at least one of a side wall of the hollow body and the at least one deflectable member, and the method may comprise detecting the presence or absence of a coil spring within the inner cavity of the hollow body by means of the or at least one of the windows. The detection of the presence or absence of a coil spring within the inner cavity of the hollow body by means of window(s) may comprise using a camera or optical sensor aligned with the window(s).

### BRIEF DESCRIPTION OF THE FIGURES

Embodiments will now be described, by way of example only, with reference to the accompanying drawings, in which:

FIG. 1 is a perspective view of a spring carrier of an embodiment of the present disclosure;

FIG. 2 is another perspective view of the spring carrier of FIG. 1, viewed from another direction;

FIG. 3 is an enlarged cross-sectional view of a region at a first end of the spring carrier of FIGS. 1 and 2, in a first position;

FIG. 4 is an enlarged cross-sectional view of a region at a first end of the spring carrier of FIGS. 1 to 3, in a second position;

FIGS. 5A-5D show a sequence of steps of use of the spring carrier of FIGS. 1-4 during insertion of a coil spring into the spring carrier;

FIGS. 6A-6D show a sequence of steps of use of the spring carrier of FIGS. 1-5D during extraction of a coil spring from the spring carrier;

FIG. 7 is a perspective view of a spring carrier and actuation plate of another embodiment;

FIG. 8A is an enlarged perspective view of an end portion of a spring carrier of another embodiment;

FIG. 8B is a cross-sectional view of the end portion of the spring carrier of FIG. 8A;

FIG. 9A is an enlarged perspective view of an end portion of a spring carrier of another embodiment;

FIG. 9B is a cross-sectional view of the end portion of the spring carrier of FIG. 9A;

FIG. 10A is an enlarged perspective view of an end portion of a spring carrier of another embodiment;

FIG. 10B is a cross-sectional view of the end portion of the spring carrier of FIG. 10A;

FIG. 11 is a schematic view of an assembly system of an embodiment of the disclosure;

FIG. 12 is a perspective view of a spring carrier of another embodiment of the disclosure;

FIG. 13 is a perspective view of a spring carrier of another embodiment of the disclosure;

FIG. 14 is a perspective view of a spring carrier of another embodiment of the disclosure;

FIG. 15 is a schematic cross-sectional view of opposing deflectable members showing retaining portions of an embodiment of the disclosure;

FIG. 16 is a schematic cross-sectional view of opposing deflectable members showing retaining portions of another embodiment of the disclosure;

FIG. 17 is a schematic cross-sectional view of a deflectable member of an alternative embodiment to that of FIGS. 15 and 16;

FIG. 18A is an enlarged view of a portion of a deflectable member of an embodiment of the disclosure showing a retaining portion of a first variant;

FIG. 18B is an enlarged view of a portion of a deflectable member of an embodiment of the disclosure showing a retaining portion of a second variant;

FIG. 18C is an enlarged view of a portion of a deflectable member of an embodiment of the disclosure showing a retaining portion of a third variant;

FIG. 19 is an enlarged cross-sectional view of a region at a first end of the spring carrier of FIGS. 1 to 3, in a second position, with an actuator of an alternative embodiment;

FIG. 20 is a perspective view of a spring carrier of another embodiment of the disclosure; and

FIG. 21 is a cross-sectional view of a portion of the spring carrier of FIG. 20.

### DETAILED DESCRIPTION

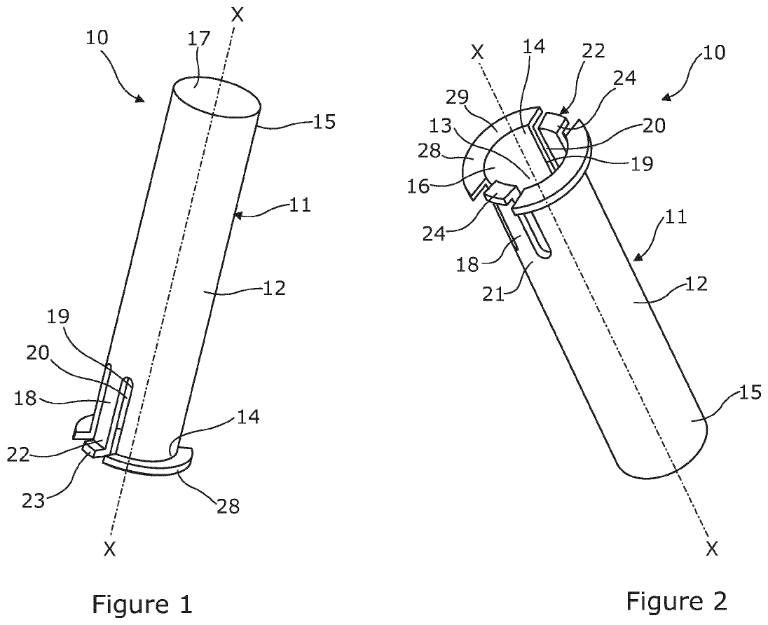

FIGS. 1 to 3 show a spring carrier 10 of an embodiment of the disclosure, and which comprises a hollow body 11 having a side wall 12 formed as a tube and defining an inner cavity 13. The hollow body 11 includes opposite first, proximal and second, distal ends 14, 15. The hollow body 11 is circular in cross-section and comprises a central axis X-X. A first opening 16 is provided at the first proximal end 14. In the exemplary embodiment, the second, distal end 15 is provided with an end wall 17 which closes the second end 15 of the hollow body 11. The inner cavity 13 is thereby accessible only via the first opening 16.

The spring carrier 10 includes two deflectable members, which in the illustrated exemplary embodiment comprise resilient arms 18. The resilient arms 18 are provided in the side wall 12 of the hollow body 11. The resilient arms 18 are disposed within apertures 19 in the side wall 12 such that a space 20 is provided around the resilient arms 18. The resilient arms 18 are joined to the side wall 12 at a respective fixed end 21 thereof. The resilient arms 18 are configured to flex about the fixed end 21. The resilient arms 18 have a free end 22 at an opposite end of the respective resilient arm 18 to the fixed end 21. The resilient arms 18 include an actuation feature for engagement with an actuator 30 (described in more detail below) operable to move the resilient arms 18 in use of the spring carrier 10. In the exemplary embodiment shown, the actuation feature comprises a lip 23 provided at the free end 22 of each resilient arm 18.

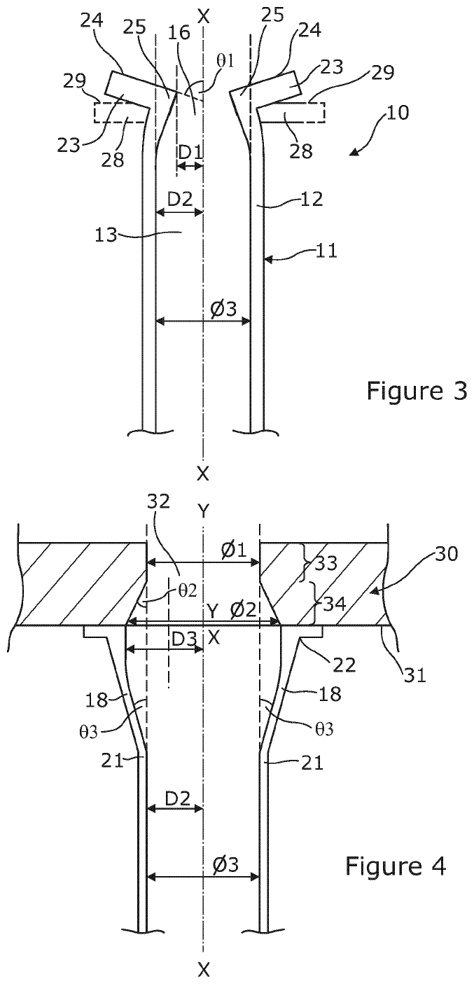

The lip 23 extends generally outwardly relative to a central axis X-X of the hollow body 11. The lip 23 comprises a contact surface 24 facing generally in an axial direction of the first end 14 of the hollow body 11. That is, the contact surface 24 generally faces away from the second end 15 of the hollow body 11. The contact surface 24 comprises a ramped surface extending at an acute angle θ1 with respect to the central axis X-X, as shown in FIG. 3. The contact surface 24 may be configured at an angle θ1 of between 10 and 30 degrees with respect to the central axis X-X, and may be between 15 and 25 degrees, for instance around 20 degrees.

The resilient arms 18 include retaining portions which are configured, in use, to retain a coil spring C within the inner cavity 13 when a coil spring C is disposed within the inner cavity 13. The retaining portions comprise a projecting region 25 extending from the respective resilient arm 18, and directed inwardly towards the central axis X-X of the hollow body 11. The projecting regions 25 are disposed at the free ends 22 of the respective resilient arm 18.

The resilient arms 18 are generally elongate and extend substantially parallel to the central axis X-X of the hollow body 11. The resilient arms 18 are substantially co-planar and/or flush with the side wall 12 of the hollow body 11. That is, in an exemplary embodiment in which the hollow body 11 is cylindrical, the resilient arms 18 substantially follow the cylindrical form of the side wall 12. The resilient arms 18 are moveable by being elastically deflected. The resilient arms 18 are in a relaxed state when in a first position, as shown in FIG. 3. In the first position, the resilient arms 18 extend substantially parallel to the central axis X-X of the hollow body 11, and substantially flush with the side wall 12 of the hollow body 11. The resilient arms 18 may be deflected away from the central axis X-X into a second position, shown in FIG. 4. The resilient arms are elastically deformed in the second position.

An inner-most portion of the projecting regions 25 may be disposed radially inwardly of the plane of the inner surface of the side wall 12 of the hollow body 11 when the respective resilient arm 18 is in the first, relaxed position, as shown in FIG. 3. This can be seen, for example, in FIG. 3, in which a distance D1 between an inner most portion of the projecting region 25 and the central axis X-X is less than a distance D2 between the inner surface of the side wall 12 of the hollow body 11 and the central axis X-X. For instance, the projecting region 25 is disposed further radially outwardly from the central axis X-X than the inner surface of the side wall 12 of the hollow body 11 in the second, elastically deformed position of the resilient arms 18. This can be seen, for example, in FIG. 4, in which a distance D3 between an inner most portion of the projecting region 25 and the central axis X-X is greater than the distance D2 between the inner surface of the side wall 12 of the hollow body 11 and the central axis X-X. This configuration will permit a coil spring C to be insertable into the inner cavity 13 when the resilient arms 18 are in the second, deformed position and the projecting regions 25 to be clear of the coil spring C, but the coil spring C to be unable to pass beyond the projecting regions 25 and thereby be retained within the inner cavity 13 when the resilient arms 18 are in the first, relaxed position.

A flange 28 is provided on the outer surface of the hollow body 11 and extends radially outwardly in a direction perpendicular to the central axis X-X. The flange 28 includes an abutment surface or stop surface 29 facing in an axial direction of the first end 14 of the hollow body 11. That is, the stop surface 29 generally faces away from the second end 15 of the hollow body 11. In the exemplary embodiment shown, the flange 28 is located at the proximal-most region of the first proximal end of the hollow body 11.

In the relaxed, first position of the resilient arms 18, the lips 23 extend further an axial direction of the first end 14 of the hollow body 11 than the flange 28 and stop surface 29. That is, the lips 23 are disposed axially further away from the second end 15 of the hollow body 11 than the flange 28 and its stop surface 29. This can be seen, for example, in FIG. 3.

In the deflected, second position of the resilient arms 18, the lips 23 are substantially level with the flange 28 with respect to an axial direction of the hollow body 11. That is, the contact surface 24 of the lips 23 is substantially level, co-planar or flush with the stop surface 29 of the flange 28 with respect to an axial direction X-X of the hollow body 11. This can be seen, for example, in FIG. 4. In such position, the contact surfaces 24 and stop surface 29 are disposed substantially perpendicular with respect to the central axis X-X of the hollow body 11.

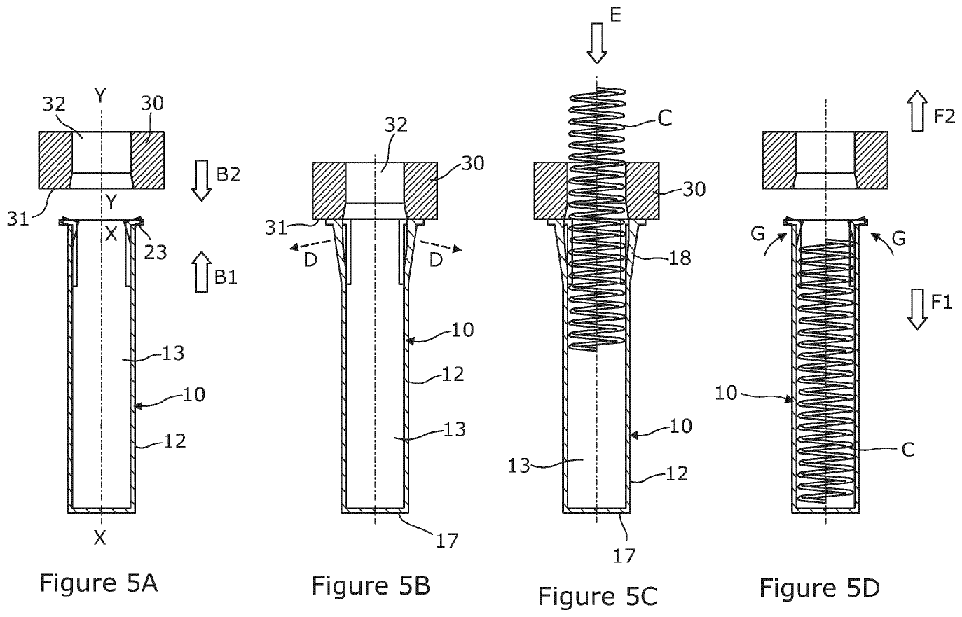

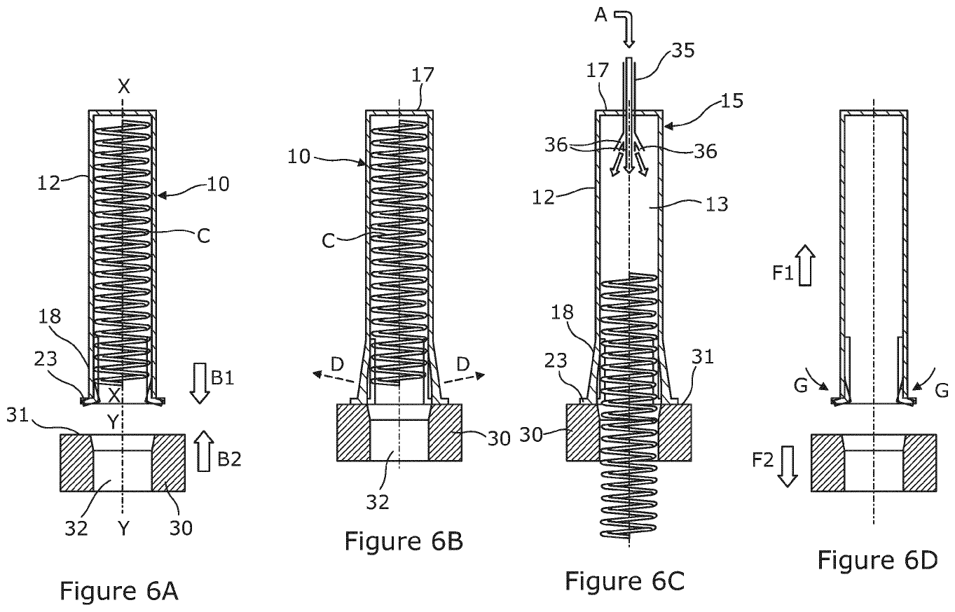

In use during a manufacturing and assembly process, the spring carrier 10 is used to receive, retain, convey, and discharge a coil spring C. Such a manufacturing process may include, for example, a method of manufacturing a medicament delivery device in which a coil spring C may be included as a biasing member to actuate a medicament delivery mechanism or to actuate a needle safety mechanism after a medicament has been delivered. Use of the spring carrier 10 will now be described with reference to FIGS. 5A-5D and FIGS. 6A-6D.

The spring carrier 10 is intended to be used in conjunction with an actuator 30 operable to move the resilient arms 18. The actuator 30 and spring carrier 10 may comprise two components of an apparatus of the present disclosure. Such apparatus may comprise a spring carrier apparatus and may comprise part of an assembly system or apparatus for a medical device, and may comprise part of an assembly and/or manufacturing apparatus/system for a medicament injection device. However, the disclosure is not intended to be limited to the medical device field and is applicable to any technical field in which one or more springs may be required to be handled and conveyed.

The actuator 30 comprises an actuation plate 30 comprising a planar surface 31. A feed aperture 32 is provided extending through the actuation plate 30, and includes a central axis Y-Y. In the exemplary embodiment shown, the feed aperture 32 has a cross-sectional dimension substantially the same as the cross-sectional dimension of the inner cavity 13 of the hollow body 11. In the exemplary embodiment in which the hollow body 11 is substantially cylindrical, and the feed aperture 32 is substantially circular in cross-section, then in one such embodiment, the internal diameter Ø1 of the feed aperture 32 is substantially the same as the internal diameter Ø3 of the inner cavity 13 (see FIGS. 3 and 4). This may allow improved insertion and extraction of a coil spring through the actuation plate 30 into and out of the spring carrier 10 by helping to avoid the coil spring catching on internal edges of components.

Also in the exemplary embodiment shown, the feed aperture 32 comprises a first region 33 of a substantially constant cross-sectional dimension, which, in the example of a circular cross-sectional feed aperture 32, is shown with the internal diameter Ø1. The feed aperture 32 comprises a second region 34 extending between the first region 33 and the planar surface 31 of the actuation plate 30. The second region 34 tapers outwardly with respect to the central axis Y-Y of the feed aperture 32 from the first region 33 to the planar surface 31. This is shown in FIG. 4, and in the example of a circular cross-sectional feed aperture 32, can be seen the internal diameter of the feed aperture increases from Ø1 where the second region 34 meets the first region 33, to a second, larger diameter Ø2 where the second region 34 meets the planar surface 31.

An angle θ2 by which the second region 34 of the feed aperture 32 tapers relative to the central axis Y-Y of the feed aperture, may be between 10 and 30 degrees, and may be between 15 and 25 degrees, for instance around 20 degrees.

FIGS. 5A-5D show method steps of insertion of a coil spring C into the spring carrier 10. In the first step shown in FIG. 5A, the inner cavity 13 of the spring carrier 10 is empty and the resilient arms 18 are in the first, relaxed position. Then, the first end 14 of the spring carrier 10 is presented towards the planar surface 31 of the actuation plate 30. That is, the spring carrier 10 may be moved towards the actuation plate 30 (shown by arrow B1), or the actuation plate 30 may be moved towards the spring carrier 10 (shown by arrow B2), or the spring carrier 10 and the actuation plate 30 may be moved towards each other (arrows B1 and B2). The spring carrier 10 and actuation plate 30 are relatively moved towards each other until reaching the position shown in FIG. 5B.

In the step shown in FIG. 5B, the contact surfaces 24 of the lips 23 have contacted the planar surface 31 of the actuation plate 30 and the spring carrier 10 and actuation plate 30 have been moved together until the stop surface 29 of the flange 28 abuts the planar surface 31 of the actuation plate 30. This has caused the resilient arms 18 to be elastically deflected radially outwardly to the second, deflected position, shown by arrows D in FIG. 5B. The resilient arms 18 flex about their respective fixed ends 21. The contact surfaces 24 of the lips 23 lie flush against the planar surface 29 of the actuation plate 30 in the second position of the resilient arms 18. The projecting regions 25 of the resilient arms 18 are therefore also moved outwardly with the resilient arms 18, and outwardly of an axial projection of the internal surface of the side walls 12 of the hollow body 11 defining the inner cavity 13. In the position shown in FIG. 5B, central axis Y-Y of the feed aperture 32 of the actuation plate 30 is aligned and coaxial with the central axis X-X of the hollow body 11.

In the next step shown in FIG. 5C, the coil spring C is inserted in direction shown by arrow E into the inner cavity 13 through the feed aperture 32 in the actuation plate 30 and through the first opening 16 at the first end 14 of the hollow body 11. The coil spring C is sized with an outer diameter slightly smaller than the inner diameter Ø3 of the inner cavity 13. As the resilient arms 18 are in a radially outward deflected position, the projecting regions 25 on each resilient arm 18 are clear of the coil spring C as it is inserted into the inner cavity 13 and allow the coil spring C to fall into the inner cavity 13 until it abuts the end wall 17 at the second end of the hollow body 11 as shown in FIG. 5D. The alignment of the central axis Y-Y of the feed aperture 32 of the actuation plate 30 with the central axis X-X of the hollow body 11 helps allow the coil spring C to be fed cleanly into the inner cavity 13 without catching or snagging on a part of the spring carrier 10.

In step 5D, the first end 14 of the spring carrier 10 is moved away from the planar surface 31 of the actuation plate 30. That is, the spring carrier 10 may be moved away from the actuation plate 30 (shown by arrow F1), or the actuation plate 30 may be moved away from the spring carrier 10 (shown by arrow F2), or the spring carrier 10 and the actuation plate 30 may be moved away from each other (arrows F1 and F2). This moves the planar surface 31 of the actuation plate 30 out of engagement with the contact surfaces 24 of the lips 23 of the resilient arms 18 and so the resilient arms 18 then move back to their first, rest position due to the elastic recovery of the material of the resilient arms 18, as shown by arrows G in FIG. 5D. As the resilient arms 18 reach the first, relaxed position, the projecting regions 25 extend inwardly of an axial projection of the internal surface of the side walls 12 of the hollow body 11 defining the inner cavity 13, and particularly, inwardly of a maximum external radial dimension of the coil spring C with respect to the central axis X-X of the inner cavity 13. The coil spring C is thereby securely retained within the spring carrier 10 and prevented from being able to pass back out through the first opening 16 in the first end 14 of the hollow body 11 by the resilient arms 18 and the projecting regions 25. The coil spring C can be conveyed within the spring carrier 10 to a location and manufacturing/assembly apparatus where the coil spring C is to be utilized.

The extraction process of the coil spring C from the spring carrier 10 will now be described with reference to FIGS. 6A-6E. Before the extraction process starts, and at a preceding step in the assembly or manufacturing process including the coil spring C, the spring carrier 10 is inverted from the orientation shown in the insertion method steps, so that the spring carrier 10 is oriented with the first end 14 lowermost and the second end 15 uppermost. The spring carrier is also positioned directly above a location where the coil spring C is to be deposited for the respective assembly/manufacturing process. For instance, the spring carrier 10 may be aligned vertically for the extraction process. This may help the coil spring C be extracted consistently and squarely from the spring carrier 10, that is, in a direction aligned with the central axis X-X of the hollow body 11.

The extraction process is generally the reverse of the insertion process described above. The spring carrier 10 is oriented substantially vertically with the first end 14 lowermost, and the spring carrier 10 disposed above an actuation plate 30. In the first step shown in FIG. 6A, the first end 14 of the spring carrier 10 is moved towards the actuation plate 30 (arrow B1), or the actuation plate 30 is moved towards the spring carrier 10 (arrow B2), or the spring carrier 10 and the actuation plate 30 are moved towards each other (arrows B1 and B2). The spring carrier 10 and actuation plate 30 are relatively moved towards each other until reaching the position shown in FIG. 6B. The central axis Y-Y of the feed aperture 32 is aligned and coaxial with the central axis X-X of the hollow body 11.

In the next step shown in FIG. 6B, the contact surfaces 24 of the lips 23 have contacted the planar surface 31 of the actuation plate 30 and the spring carrier 10 and actuation plate 30 have been moved together until the stop surface 29 of the flange 28 abuts the planar surface 31 of the actuation plate 30. The resilient arms 18 are elastically deflected outwardly to the second, deflected position, shown by arrows D in FIG. 6B, until the contact surfaces 24 lie flush against the planar surface 29. The projecting regions 25 of the resilient arms 18 are moved outwardly sufficiently that they no longer block the coil spring C from being able to pass out of the first opening 16 in the first end 14 of the hollow body 11.

In the next step shown in FIG. 6C, the coil spring C is free to fall under its own weight out of the inner cavity 13 through the first opening 16 at the first end 14 of the hollow body 11 and through the feed aperture 32 in the actuation plate 30. The coil spring C falls into the required location outside the spring carrier 10 and actuation plate 30 so that the coil spring C is fully extracted from the spring carrier 10. In this step, the second, tapered region 34 of the feed aperture 32 may help guide the coil spring C into the first region 33 of the feed aperture 32 as it is extracted from the inner cavity 13. This can help compensate for misalignment between the spring carrier 10 and the actuation plate 30 in the process, and/or the positional tolerance of the coil spring C within the inner cavity 13 relative to the feed aperture 32.

In step 6D, the spring carrier 10 is moved away from the actuation plate 30 (arrow F1), or the actuation plate 30 is moved away from the spring carrier 10 (arrow F2), or the spring carrier 10 and the actuation plate 30 are moved away from each other (arrows F1 and F2). The planar surface 31 of the actuation plate 30 moves out of engagement with the contact surfaces 24 and so the resilient arms 18 then move back to their first, rest position due to the elastic recovery of the material of the resilient arms 18, as shown by arrows G in FIG. 6D.

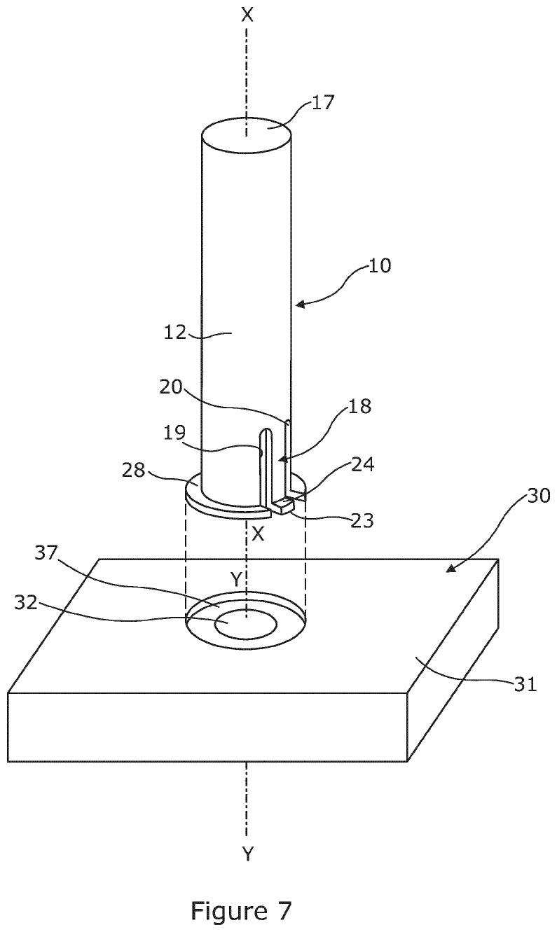

In both the insertion process and extraction process, the spring carrier 10 may be accurately aligned with the location from and to which the coil spring C is to be inserted/extracted to allow the coil spring C to be effectively conveyed as desired and not snag on ends of the spring carrier 10 or apparatus into which the coil spring C is to be discharged. In this way, manufacturing errors and/or production stoppages to correct the errors can be reduced or avoided. An alternative embodiment of actuation plate 30 configured to assist accurate spring carrier 10 alignment is shown in FIG. 7. Like features with the embodiment described above retain the same reference numerals and a detailed description thereof will not be repeated. The flange 28 may help avoid such misalignment problems by provided a locating guide for the spring carrier 10 in use. In the exemplary alternative embodiment shown in FIG. 7, the actuation plate 30 includes a recess 37 which is shaped to correspond to, and receive, the flange 28 of the spring carrier 10. This location feature can help towards ensuring the central axis X-X of the hollow body 11 is coaxial with the central axis Y-Y of the feed aperture 32, and that the coil spring C may be accurately inserted into and/or extracted from the spring carrier 10 through the feed aperture 32.

In the embodiments described above, and as shown in FIG. 4, when the actuation plate 30 engaged with the spring carrier 10, the resilient arms 18 in the second, deflected position, are deflected radially outwardly by an angle θ3 from the relaxed position in which the resilient arms 18 lie substantially parallel with the central axis X-X of the hollow body 11 and substantially flush with the side wall 12 of the hollow body 11. Angle θ3 can vary within the scope of the disclosure, and/or within any embodiment of the disclosure described herein, and may vary depending on various dimensions, such as length of resilient arms 18, distance the projecting regions 25 extend inwardly from resilient arm 18, diameter of hollow body 11, etc. However, the angle θ3 may be around 10 degrees, and may be between 4-20 degrees, for instance between 5-15 degrees. This may allow sufficient deflection of the resilient arms 18 to achieve the above-described function, without excessively fatiguing the material of the resilient arms 18 and/or hollow body 11. That is, the resilient arms 18 are able to be repeatedly elastically deflected and return to the same relaxed position, without reaching the ductile limits of the material of the spring carrier 10 which would affect the ability for the resilient arms 18 to restore themselves to the intended, first, relaxed position. Contributing factors for the desired resilient performance of the resilient arms 18 include dimensions of the length, thickness and width of the resilient arms 18, and also the elastic modulus, elasticity limit and tenacity (deformation resistance at speed) of the resilient arms. Within any embodiment of the disclosure described herein, a range of elastic modulus may be between 1800-2500 MPa, and an elasticity limit may be 40-80 MPa. Furthermore, a tenacity of the resilient arms 18 may be 150-300 J/m². In use, to allow repeated elastic deformation and limit the aging effect on the material of the resilient arms 18, the arms may be deflected only to 40-80% of the maximum elastic limit.

From FIGS. 3 and 4, a lateral outward deflection distance the resilient arms 18 deflect outward in the deflected position from the relaxed position can be seen as the distance difference (D3-D1). That is, the distance the radially inner-most portion of the projecting region 25 moves between the first unbiased position and the second biased position of the resilient arm 18. Such deflection distance may vary within the scope of the disclosure, and/or within any embodiment of the disclosure described herein, but may be between 1 mm-5 mm, and may be between 1 mm-3 mm, and may be between 1-2 mm, and may be around 1.5 mm.

In optional variants of the above-described spring carrier 10 and spring carrier apparatus, means for facilitating extraction of the coil spring C from the spring carrier 10 may be provided. Such variants will be described with reference to FIGS. 6C, and FIGS. 8A-10B. In the above-described extraction process with reference to FIGS. 6A-6D, at the step shown in step 6C, once the resilient arms 18 are in a radially outward deflected position and the projecting regions 25 on each resilient arm 18 are clear of the coil spring C, the coil spring C falls under its own weight out of the inner cavity 13 through the first opening 16 at the first end 14 of the hollow body 11. In a variant of the above-described apparatus, illustrated schematically in FIG. 6C, the apparatus may include an airflow source or air jet A to generate a flow of air through the inner cavity 13 to blow the coil spring C out of the spring carrier 10. The airflow source A may comprise an air duct or air passage 35 which may be disposed through or connected to the second end 15 of the hollow body 11. Such air passage 35 may be insertable into or disposed through the end wall 17 of the spring carrier 10. The air passage 35 may be connected or connectable to a source of pressurized air A. In use, the air source A may be connected, or turned on, as the actuation plate 30 moves the resilient arms 18 into the deflected position, to send a flow of air (shown by arrows A in FIG. 6C) through the air passage 35 and into the inner cavity 13 of the hollow body 11. The air flow A may then impinge upon the coil spring C and force the coil spring C out of the spring carrier 10.

A plurality of air passages 35 may be provided, including a plurality of air passage outlets 36. The air passage(s) 35 and/or air flow outlets 36 may be aligned substantially parallel with the central axis X-X of the hollow body 11 in use. In addition, or alternatively, one or more air flow outlets 36 and/or air flow passages 35 may be oriented at an angle with respect to the central axis X-X of the hollow body 11 in use. In the latter case, the angled air flow outlets 36/passages 35 may encourage the air flow to impinge on the coils of the coil spring C to encourage expulsion of the coil spring C from the spring carrier 10. In an embodiment in which a central axial air flow passage 35/outlet 36 is provided, turbulence of air flow through the coil spring may still cause sufficient impinging of the air flow on the coils of the coil spring C to encourage expulsion of the coil spring C from the spring carrier 10.

The air passage(s) 35 may be a separate component of an apparatus to the spring carrier 10, or may comprise a component connected to, or formed integrally with, the spring carrier 10, as will be explained in more detail below.

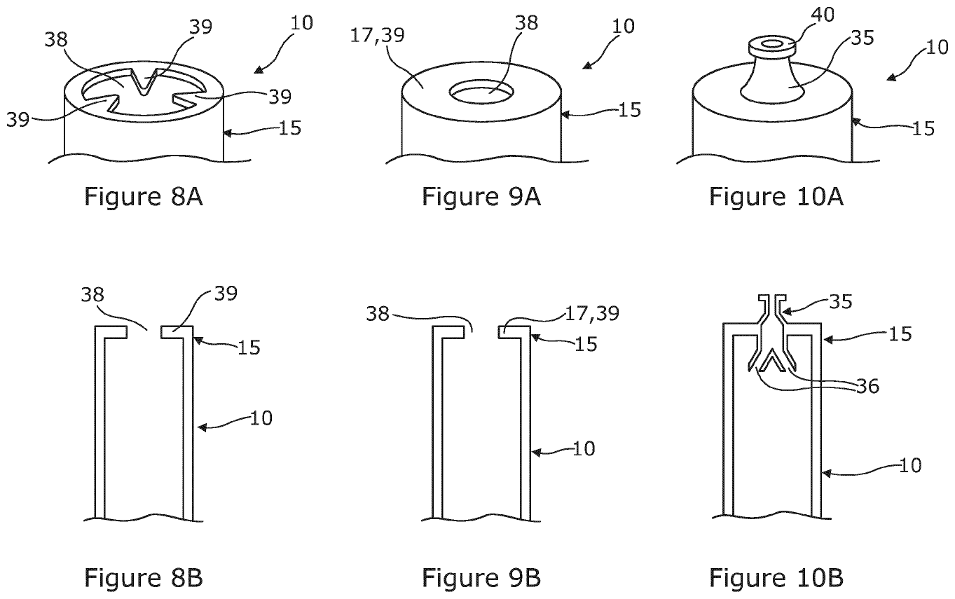

Referring to FIGS. 8A to 10B, alternative variations of spring carrier 10 are illustrated. Like features retain the same reference numerals and a detailed description thereof will not be repeated. In the embodiment of FIGS. 8A and 8B, the second end 15 of the hollow body 11 is not closed by an end wall 17 but instead includes an opening, referred to herein as a second opening 38 in the hollow body 11. Around the perimeter of the opening 38 are a plurality of protrusions 39 extending radially inwardly. In use, the protrusions 39 serve as spring stops for the coil spring C to rest against when being inserted into the spring carrier 10 (as described above with reference to FIGS. 5C and 5D). The second opening 38 may be used as an access aperture through which an airflow duct or air passage 35 may extend in variants of the disclosure in which an airflow source A is used to assist extraction of the coil spring C from the spring carrier 10, as described above with reference to FIG. 6C.

In the embodiment of FIGS. 9A and 9B, the second end 15 is not entirely closed by the end wall 17. Instead, the end wall 17 includes an opening, referred to herein as a second opening 38. Or, considered alternatively, the end wall 17 is a single continuous protrusion 39 extending radially inwardly around the perimeter of the second end 15 of the hollow body 11 but not closing the second end 15, to leave the second opening 38. In use, the end wall 17/inward protrusion 39 serves as a spring stop for the coil spring C to rest against when being inserted into the spring carrier 10 (as described above with reference to FIGS. 5C and 5D). The second opening 38 may be used as an access aperture through which an airflow duct or air passage 35 may extend in variants of the disclosure in which an airflow source A is used to assist extraction of the coil spring C from the spring carrier 10, as described above with reference to FIG. 6C.

In the embodiment of FIGS. 10A and 10B, the second end 15 is not closed by an end wall 17 but instead, the end wall 17 includes an airflow duct 35 extending through the end wall 17. The airflow duct 35 may be a separate component secured to the end wall 17, such as by bonding, mechanical fastening, welding or other known means. Alternatively, the airflow duct 35 may be formed integrally with the end wall 17 and the hollow body 11. The airflow duct 35 includes a plurality of outlets 36 within the inner cavity 13. The outlets 36 are oriented at an acute angle other than perpendicular or parallel to the central axis X-X of the hollow body. This may help provide the advantages mentioned above. However, in alternative embodiments, there may be only one outlet 36 or more than two outlets 36, and/or the or each outlet may be oriented substantially parallel with the central axis X-X of the hollow body 11. The airflow duct 35 includes an inlet 40. In use, pressurized airflow A may be fed into the airflow duct 35 through the inlet 40. The inlet may be configured with a connection for coupling the airflow duct 35 to an airflow source A.

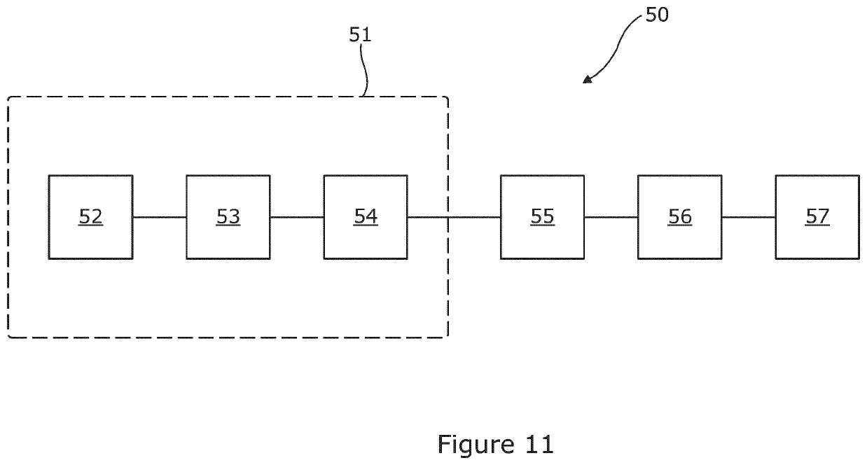

The spring carrier 10, and apparatus comprising the spring carrier 10 and actuation plate 30, may be part of a larger assembly system or apparatus for manufacturing devices which include one or more coil springs C. Such system may comprise a plurality of assembly machines or stations. Such assembly machines/stations may be configured as an inline process and as two or more separate processes. An exemplary assembly system 50 is shown schematically in FIG. 11. The assembly system 50 includes a coil spring making system, generally designated 51. The coil spring making system 51 may include a coiling station 52 which creates the coil spring C, a heating station 53 where the coiled spring is heated to temper the material of the spring. The heated coiled spring is then fed to a cooling station 54 to cool the coiled spring. Thereafter, a conveyor 55 transfers the cooled coiled spring C to an insertion station 56 which includes the apparatus comprising the actuation plate 30 and with which the spring carrier 10 may be provided. At the insertion station 56, the actuation plate 30 and spring carrier 10 are operated as described above to insert the coil spring C into the spring carrier 10. The spring carrier 10 with coil spring retained therein is conveyed to an extraction station 57. At the extraction station 57, the actuation plate 30 and spring carrier 10 are operated as described above to extract the coil spring C from the spring carrier 10 for use in subsequent device assembly steps in which the coil spring C is utilized.

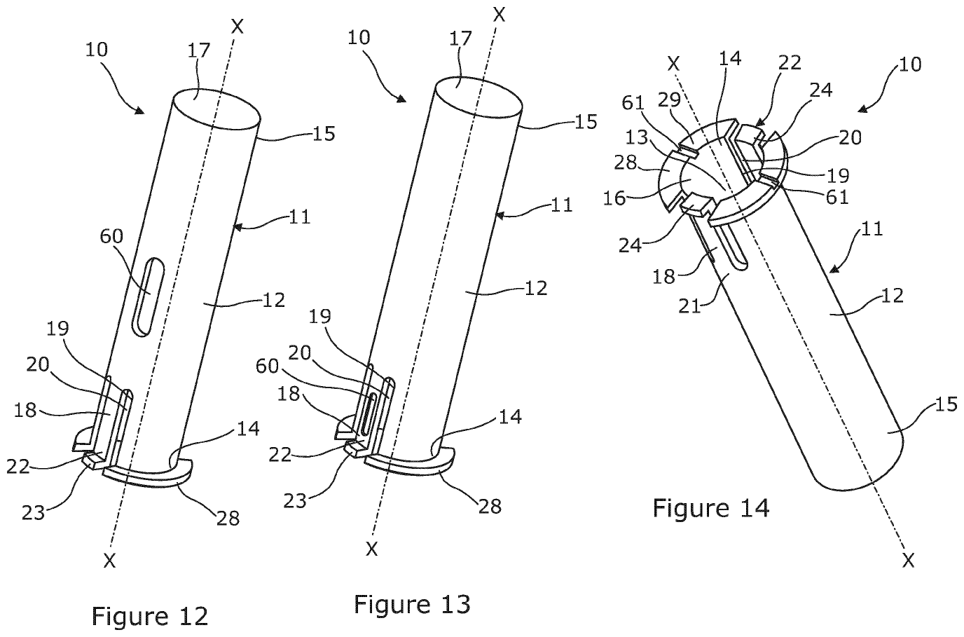

FIG. 12 shows a spring carrier 10 of another embodiment of the disclosure, similar to that shown in FIG. 1, and like features retain the same reference numerals and detailed description thereof will not be repeated. A difference with the embodiment of FIG. 12 is that a window or cut-out region 60 is provided in and extending through the side wall 12 of the hollow body 11. This enables the interior of the hollow body 11 to be viewed from the outside of the spring carrier 10. Particularly, this enables a coil spring C to be seen when received within the spring carrier 10. This may benefit use of the spring carrier 10 in a manufacturing process. For example, in a quality control or performance monitoring process, the presence of a coil spring C within the spring carrier 10 may be checked for each device being produced. For example, an optical sensor or camera may check for the presence of a coil spring C within the spring carrier 10 and may operate using the window 60 to make such checks. For example, if it is detected that a coil spring C is absent from the spring carrier 10 due to an insertion fault elsewhere in the manufacturing process, the device being produced will likely not function correctly without the coil spring C and so can be automatically rejected from the production line. One window 60 may be provided, or a plurality of windows may be provided, and maybe disposed in any suitable location on the side wall 12 of the spring carrier 10. The window(s) 60 also mean that less material is required to manufacture each spring carrier 10, which may reduce cost of manufacture and/or may also reduce the weight of the spring carrier which may be beneficial in the device manufacturing processes in which the spring carrier is to be used.

FIG. 13 shows a spring carrier 10 of another embodiment of the disclosure, similar to that shown in FIG. 12, and like features retain the same reference numerals and detailed description thereof will not be repeated. A difference with the embodiment of FIG. 13 is that although a window 60 is provided, it is provided in the resilient arm 18 instead of the side wall 12 of the hollow body 11. The window 60 still provides the advantage described above of being able to detect the presence of a coil spring C within the spring carrier 10. However, the window 60 may also render the resilient arm 18 lighter and/or more readily deflectable than if the window 60 was not provided in the resilient arm 18. This may require less actuator force to deflect the resilient arm 18 by the required amount in use of the spring carrier 10. Such reduced forces may reduce the stress on the material of the spring carrier 10 and enable greater life cycle of the spring carrier 10 before failure or replacement is required.

FIG. 14 shows a spring carrier 10 of another embodiment, similar to the embodiment of FIGS. 1, 12 and 13, and like features retain the same reference numerals and detailed description thereof will not be repeated. A difference with the embodiment of FIG. 14 is that the flange 28 includes orientation features 61. In the exemplary embodiment shown, the orientation features 61 comprise a pair of radial slots formed into the surface of the flange 28 facing in the direction of the first end 14. Such orientation features 61 may facilitate correct rotational positioning of the spring carrier 10 about its central axis X-X which may be beneficial for function of the spring carrier 10 in use, for example for insertion or extraction of a coil spring C. Furthermore, such orientation feature may be used in conjunction with the window 60 during a manufacturing process. For example, an optical sensor or camera used to detect the presence of a coil spring C within the spring carrier 10 may be located in a certain position on a manufacturing apparatus/system or assembly line, and so require correct orientation of the spring carrier 10 to align the window 60 with the optical sensor or camera. The orientation features 61 may cooperate with corresponding features (not shown) such as projections which may be received in the slots of the orientation features 61 to ensure correct positioning of the spring carrier 10 in use.

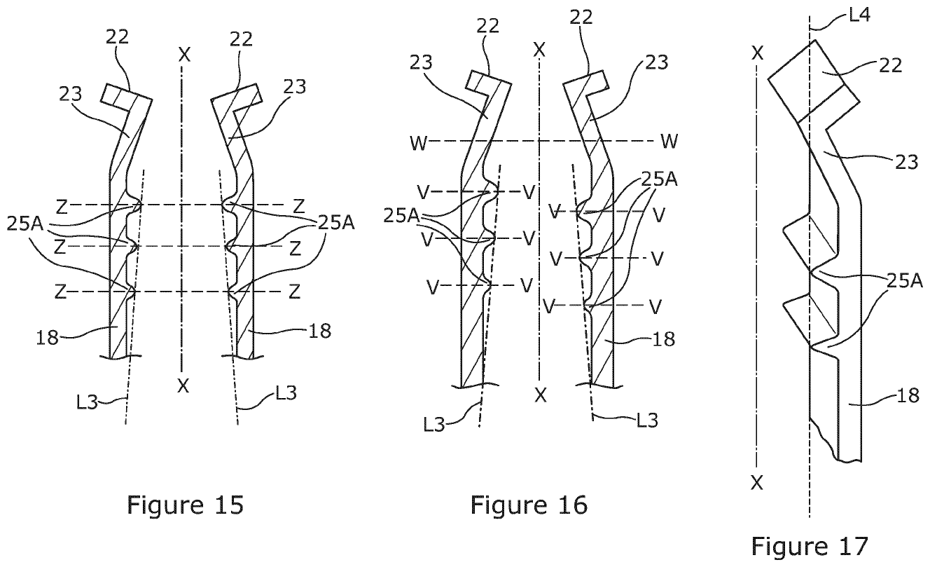

The configuration and arrangement of retaining portions on the resilient arms 18 may vary within the scope of the disclosure, and such variants intended within the scope of the disclosure, and/or within the scope of all embodiments described herein, are illustrated as non-exhaustive examples in FIGS. 15 to 17. Such retaining portions may include features in addition to the projecting regions 25, as will be described hereafter.

FIG. 15 shows a schematic cross-sectional view of a configuration of one embodiment, showing only the opposing resilient arms 18 in a relaxed state, that is, the first unbiased position. Each resilient arm comprises a projecting region 25 and additionally a plurality of projecting elements 25A spaced in an axial direction of each resilient arm 18. Although a plurality of projecting elements 25A are shown on each resilient arm 18, each resilient arm 18 may comprise only one projecting element 25A. The projecting elements 25A are configured, in use, to engage with a coil spring C when a coil spring C is disposed within the inner cavity 13 and to further assist retaining the coil spring C in place within the inner cavity 13. Each projecting element 25A extends from the respective resilient arm 18, and is directed inwardly towards the central axis X-X of the hollow body 11.

In use, an inner-most portion of the projecting elements 25A may be disposed radially inwardly of the plane of the inner surface of the side wall 12 of the hollow body 11 when the respective resilient arm 18 is in the first, relaxed or unbiased position, to engage and retain a coil spring within the inner cavity 13 of the hollow body 11. The projecting elements 25A may be disposed radially outwardly of the plane of the inner surface of the side wall 12 of the hollow body 11 when the respective resilient arm 18 is in the second, deflected or biased position, to allow a coil spring to be inserted into or extracted from the inner cavity 13 of the hollow body 11.

In the embodiment in FIG. 15, the projecting elements 25A of one resilient arm 18 are aligned in an axial direction of the spring carrier 10 with the corresponding projecting elements 25A of the opposing resilient arm 18. This is shown by reference lines Z-Z, which extend through each projecting element 25A of one resilient arm 18 in a direction perpendicular to the axis X-X of the spring carrier 10, extending through the corresponding projecting element 25A on the opposite resilient arm 18. This may help securely retain a coil spring C with minimal axial movement by encouraging opposing projecting elements 25A to abut against and clamp a region of the coil spring C when retained within the spring carrier 10. This arrangement may be applicable in embodiments of spring carrier 10 of the disclosure which comprise two resilient arms 18, or more than two resilient arms 18.

In the embodiment of FIG. 15, the plurality of projecting elements 25A on each resilient arm 18 increase in size incrementally towards the free, proximal end 22 of each resilient arm 18. That is, the distance each projecting element 25A projects inwardly towards the central axis X-X of the spring carrier 10 is greater the closer to the free proximal end 22 of the resilient arm 18 each is located. This is shown by lines L3 aligned with the inner-most portion of each projecting element 25A being angled inwardly towards the central axis X-X in a direction towards the free proximal ends 22 of the resilient arms 18. This may help securely retain a coil spring C within the spring carrier 10, as larger, more inwardly-extending projecting elements 25A may be provided towards the free ends 22 of the resilient arms, yet as the free ends 22 are deflected laterally outwardly by a greater distance than a region of each resilient arm 18 spaced from the free end 22 when actuated by the actuator 30 as described above, the larger projecting elements 25A are still moved sufficiently outwardly to permit insertion or extraction of the coil spring C.

FIG. 16 shows a schematic cross-sectional view of a configuration of another embodiment, similar to that of FIG. 15, and in which like features retain the same reference numerals. The opposing resilient arms 18, each comprising a projecting region 25 and a plurality of projecting elements 25A spaced in an axial direction of each resilient arm 18. A difference in the embodiment of FIG. 16 is that the projecting elements 25A of one resilient arm 18 are not aligned in an axial direction of the spring carrier 10 with the corresponding projecting elements 25A of the opposing resilient arm 18 but instead are off-set in an axial direction of the spring carrier 10 with respect to the corresponding projecting elements 25A of the opposing resilient arm 18. This is shown by reference lines V-V, which extend through each projecting element 25A of one resilient arm 18 in a direction perpendicular to the axis X-X of the spring carrier 10, not being aligned with those lines V-V through the corresponding projecting element 25A on the opposite resilient arm 18. This may help securely retain a coil spring C with minimal axial movement, and/or with axial alignment, by the staggered opposing projecting elements 25A following the helical coil of the coil spring C when retained within the spring carrier 10. This arrangement may be applicable in embodiments of spring carrier 10 of the disclosure which comprise two resilient arms 18, or more than two resilient arms 18.

In the embodiment of FIG. 16, the plurality of projecting elements 25A on each resilient arm 18 increase in size incrementally towards the free end 22 of each resilient arm 18, as described above with reference to FIG. 15. This is shown in FIG. 16 by lines L3 aligned with the inner-most portion of each projecting element 25A being angled inwardly towards the central axis X-X in a direction towards the free proximal ends 22 of the resilient arms 18. This may provide the same advantages described above with reference to FIG. 15.

FIG. 17 is a schematic view of a configuration of a resilient arm 18 of another embodiment, and is similar to those of FIGS. 15 and 16. The embodiment of FIG. 17 differs in that the plurality of projecting elements 25A on each resilient arm 18 are of the same size. That is, the distance each projecting element 25A projects inwardly towards the central axis X-X of the spring carrier 10 is the same. This is shown by line L4 aligned with the inner-most portion of each projecting element 25A being parallel with the central axis X-X of the spring carrier 10. This may help securely retain a coil spring C within the spring carrier 10, as in the first, relaxed or unbiased position of the resilient arms 18, each projecting element 25A projects equally to engage and secure a coil spring C within the spring carrier 10.

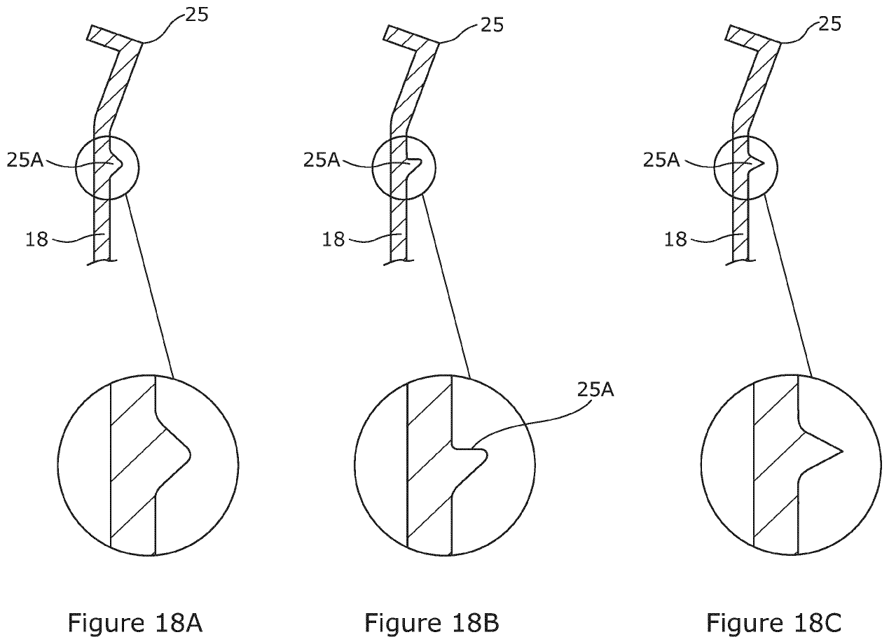

FIGS. 18A to 18C are schematic enlarged views of resilient arms 18 of spring carriers 10 of embodiments of the disclosure, showing different configurations of projecting elements 25A intended to fall within the scope of the disclosure and applicable to all embodiments described herein. FIG. 18A shows a projecting element 25A comprising a generally rounded shape, with curved edges where the projecting element 25A extends from the resilient arm 18, and at the axially inner-most region of the projecting element 25A. Such configuration may facilitate insertion of a coil spring C into the spring carrier 10, for example by allowing a coil spring C to ride over the projecting elements 25A if it should contact them during insertion when the resilient arms 18 are deflected outwardly, to allow the coil spring C to move to the fully inserted position.

The projecting element 25A of FIG. 18B is arranged with one surface 25B extending substantially perpendicular to the resilient arm 18 and to the axis X-X of the spring carrier 10. It is intended that such a surface 25B may be provided facing either the first end 14, or second end 15 of the spring carrier 10 within the scope of the disclosure. Yet further, within the scope of the disclosure, the projecting elements 25A may be configured with two such surfaces 25B extending substantially perpendicular to the resilient arm 18 and to the axis X-X of the spring carrier 10, one surface 25B facing the first end 14 and a second such surface 25B facing the second end 15 of the spring carrier 10. Such configuration may facilitate retention of a coil spring C within the spring carrier 10 in a desired axial position, as axial movement of the coil spring C would be more limited due to the perpendicular shape of the surfaces 25B.

FIG. 18C shows a projecting element 25A comprising a generally angled shape, with straight edges meeting at an angle where the projecting element 25A extends from the resilient arm 18, and at the axially inner-most region of the projecting element 25A. Such configuration may facilitate engagement of a coil spring C within the spring carrier 10, for example by allowing the pointed edges of the projecting elements 25A to more easily locate between coils of a coil spring C when the resilient arms 18 are released by the actuator to return to their relaxed position.

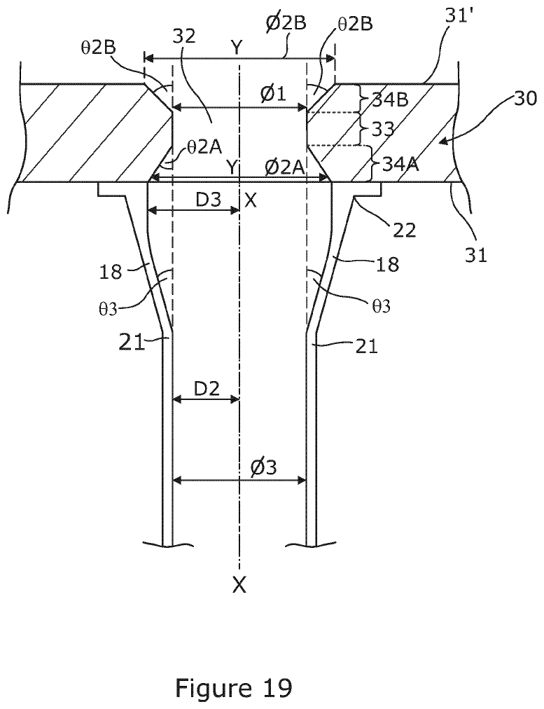

FIG. 19 is similar to FIG. 4, and shows a spring carrier and actuator 30 comprising an actuation plate of another embodiment, and like features with those of FIG. 4 retain the same reference numerals. As with the embodiment of FIG. 4, the feed aperture 32 comprises a first region 33 of a substantially constant cross-sectional dimension shown with the internal diameter Ø1, and a second region, indicated here as 34A, extending between the first region 33 and the planar surface 31 of the actuation plate 30, and which tapers outwardly with respect to the central axis Y-Y of the feed aperture 32 from the first region 33 to the planar surface 31. This is shown in FIG. 19 by the internal diameter of the feed aperture 32 increases from Ø1 where the second region 34A meets the first region 33, to a second, larger diameter Ø2A where the second region 34A meets the planar surface 31.

A difference with the embodiment of FIG. 19 is that the actuation plate 30 comprises a third region 34B extending between the first region 33 and a second planar surface 31′ of the actuation plate 30 on the opposite side thereof to the first planar surface 31. The third region 34B tapers outwardly with respect to the central axis Y-Y of the feed aperture 32 from the first region 33 to the second planar surface 31′. This is shown in FIG. 19 by the internal diameter of the feed aperture 32 increases from Ø1 where the third region 34B meets the first region 33, to a second, larger diameter Ø2B where the third region 34B meets the second planar surface 31′.

As with the embodiment of FIG. 4, an angle θ2A by which the second region 34A of the feed aperture 32 tapers relative to the central axis Y-Y of the feed aperture 32, may be between 10 and 30 degrees, and may be between 15 and 25 degrees, for instance around 20 degrees. An angle θ2B by which the third region 34B of the feed aperture 32 tapers relative to the central axis Y-Y of the feed aperture 32, may be between 10 and 30 degrees, and may be between 15 and 25 degrees, for instance around 20 degrees. The angles θ2A and θ2B may be the same or may differ within the scope of the disclosure. The third region 34B may allow improved insertion of a coil spring C initially through the actuation plate 30 from the second planar surface 31′ side before the coil spring C reaches the spring carrier 10, and may help avoid the coil spring C catching on an edge of the actuation plate 30.

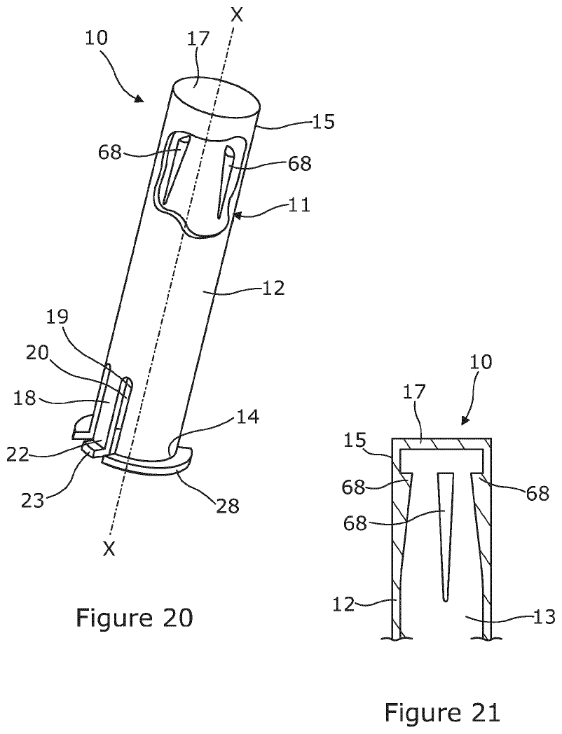

FIGS. 20 and 21 show a spring carrier 10 of another embodiment of the disclosure, similar to the spring carrier of FIGS. 1-4, and like features retain the same reference numerals and detailed description thereof will not be repeated. A difference in the spring carrier of FIGS. 20 and 21 is that the inner surface of the side wall 12 of the hollow body 11 includes a plurality of centering lugs 68 which project inwardly towards the central axis X-X of the hollow body 11. FIG. 20 shows a portion of the side wall 12 cut-away to enable these centering lugs 68 to be illustrated. In the embodiment shown, four centering lugs 68 are provided. However, more than four or fewer than four may be provided, and the centering lugs 68 may optionally be equally spaced around the inside circumference of the side wall 12.

The centering lugs 68 are formed as ramps with a curved surface and increase in the distance they project inwardly as the centering lug 68 extends towards the second distal end 15 of the spring carrier 10. In use, the centering lugs 68 serve to contact and center a coil spring C held within the spring carrier 10 so that the coil spring C is accurately retained centrally within the spring carrier 10. The centering lugs 68 may compensate for any tolerance between the outer diameter of the coil spring C and internal diameter of the inner cavity 13 to reduce play between the coil spring C and spring carrier 10. This may help ensure the coil spring C is accurately located during insertion of the coil spring C into the spring carrier 10, to help ensure the coil spring C can be securely engaged by the retaining portion(s). This may help avoid accidental or premature spring extraction during transportation of the spring carrier 10 or during a manufacturing process in which the coil spring is required to be accurately extracted and positioned into a device being manufactured. The may help prevent manufacturing errors and/or stoppages. The feature of the centering lugs 68 may optionally be applicable to and provided with any embodiment of the disclosure described herein.

The various embodiments of spring carrier 10 illustrated and described above are intended to be configured in a range of shapes and sizes and relative dimensions within the scope of the disclosure.

The spring carrier 10 may comprise a total length in a direction of the axis X-X of between 50 mm to 90 mm, and may be between 60 mm to 80 mm, and may be around 70.5 mm or around 73.5 mm.

The flange 28 may comprise a height in a direction of the axis X-X of between 1 mm to 5 mm, and may be between 2 mm to 4 mm, and may be around 3 mm.

The resilient arms 18 may comprise a total length in a direction of the axis X-X from the fixed end 21 to the free end 22 of between 10 mm to 20 mm, and may be between 12 mm to 18 mm, and may be between 14 to 16 mm, and may be around 16.3 mm.

The window 60 when provided in the side wall 12 may comprise a length in a direction of the axis X-X of between 5 mm to 25 mm, and may be between 10 mm to 20 mm, and may be around 15 mm. The window 60 when provided in the resilient arms 18 may comprise a length in a direction of the axis X-X of between 1.5 mm to 8 mm, and may be between 2.5 mm to 7 mm, and may be between 3.5 to 6 mm, may be around 4.3 mm.

The hollow body 11 is shown and described as being configured as a cylindrical tube which is circular in cross-section. This may allow the hollow body 11 to closely contain coil springs C of conventional circular form. This also may facilitate ease of insertion of the coil springs C, and alignment of the spring carrier 10 for extraction of the coil spring C, as no specific rotational orientation about the central axis X-X is required for correct positioning of the spring carrier 10 in use. However, the disclosure is not intended to be limited to such a configuration of spring carrier, and other dimensions and cross-sectional shapes are possible, such as oval, triangular or square, or other polygonal cross-sectional shapes.

The hollow body 11 is shown and described as being of a substantially constant cross-section along its length from the first end 14 to the second, opposite end 15. This may facilitate ease and cost of manufacture and of manipulation in assembly or manufacturing processes in which the spring carrier 10 is to be utilized. However, the disclosure is not intended to be limited to such a configuration and in alternative embodiments, the spring carrier 10 may vary in cross-sectional dimension along its length. For example, the cross-section may be circular of different diameters along the length of the spring carrier, and/or the cross-section may be shaped other than circular along part of the length of the spring carrier. For example, the internal diameter may be larger in the region of the first end 14 through which the coil spring C is inserted and extracted, than in the region of the second end 15. This may further help accurately guide the coil spring C into the spring carrier 10. This may also allow the coil spring C to be more closely confined in the region of the second end 15 of the spring carrier 10. However, the opposite may be the case within the scope of the disclosure and the internal diameter at the first end 14 may be smaller than the internal diameter at the second end 15 such that the inner cavity 13 is slightly narrower in the region of the first end 14 of the spring carrier 10.

In an exemplary embodiment in which the internal diameter is substantially uniform along the length of the hollow body 11, the internal diameter may be between 7 mm to 14 mm, and may be between 8 mm to 13 mm, and may be between 9 mm to 12 m, and may be between 10 mm to 11 mm, and may be around 10.5 mm or around 11.5 mm.

In an exemplary embodiment in which the internal diameter is not uniform along the length of the hollow body, the internal diameter at one end of the hollow body may be between 9 mm to 14 mm, and may be between 10 mm to 13 mm, and may be between 11 mm to 12 m, and may be around 11.5 mm. The internal diameter at the other end of the hollow body may be between 8 mm to 13 mm, and may be between 9 mm to 12 mm, and may be between 10 mm to 11 m, and may be around 10.5 mm.

Various materials may be selected from which the spring carrier 10 is formed, which includes plastics and metals, and may include various polymers, including Polypropylene, Polyester, Polyamide or Acrylo-Butadiene-Styrene (ABS). The spring carrier may further be formed from Polycarbonate, and may comprise recycled Polycarbonate.

The spring carrier 10 is shown and described as a single molded component, that is, a single integral component. As such, the resilient arms 18 for example, are shown as being integrally formed with hollow body 11. This may provide advantages of ease and reduced cost of manufacture. However, it is intended within the scope of the disclosure that one or more elements of the spring carrier 10 may be separate components secured, bonded, welded, mechanically fastened together. For example, the resilient arms 18, or the flange 28, may not be integrally formed with the hollow body 11.

The side wall 12 and end wall 17 of the hollow body 11 may be of a dimension to provide sufficient structural strength during use, but also minimize excess use of material and maintain light-weight for ease of handling and cost of manufacture. The wall thicknesses may be between 0.3 mm to 1.5 mm, and may be between 0.5 mm-1 mm in thickness.

Embodiments of spring carrier 10 and associated apparatus/systems of the present disclosure are configured to securely retain a coil spring C therein, and reliably and accurately allow extraction of the coil spring C. In order that the coil spring can be both securely retained and accurately extracted, the spring carrier 10 may be configured such that a certain clearance is provided between an outer diameter of the coil spring C and an inside wall of the inner cavity 13. The clearance may be set to allow substantially unimpeded insertion and extraction of the coil spring C into/from the inner cavity 13, yet also minimize lateral play or movement of the coil spring C within the inner cavity so that the coil spring can be accurately discharged where required. In an embodiment, such clearance may be 0.05 mm-0.3 mmm, and may be between 0.1 mm-0.2 mm. In one embodiment, coil springs C to be received in the inner cavity 13 may have a maximum outer diameter of 9.95 mm. Accordingly, an internal diameter Ø3 of the inner cavity 13 (shown in FIG. 4) may be around 10.0 mm-12.95 mm, and may be around 10.05 mm-11.05 mm.

Although the embodiments of spring carrier 10 shown and described comprise two resilient arms 18, the disclosure is not intended to be limited to this configuration and in alternative embodiments, the spring carrier 10 may comprise only one, or more than two resilient arms 18. In embodiments comprising two or more resilient arms 18, the resilient arms 18 may be equally spaced around the perimeter of the spring carrier 10 for even and aligned retaining of a coil spring C in spring carrier 10. Additionally, such a configuration may also help promote the coil spring C being extracted evenly and in axial alignment with spring carrier 10 and into a component of medical device or manufacturing apparatus, for example, as intended.

The embodiments of spring carrier 10 shown and described comprise resilient arms 18 having one projecting region 25 on each resilient arm 18. However, the disclosure is not intended to be limited to such a configuration and in alternative embodiments, a plurality of projecting regions/elements 25/25A and/or a plurality of notches may be provided on each resilient arm 18 configured such that the spring carrier 10 may engage multiple turns of a coil spring C received within the hollow body 11. Furthermore, some embodiments described above may comprise projecting elements 25A which may engage and retain a coil spring within the inner cavity 13 of the hollow body 11 when the respective resilient arm 18 is in the first, relaxed or unbiased position. It is envisaged that the projecting element(s) 25A may be sufficient to retain the coil spring within the inner cavity and restrict movement of the coil spring in the axial direction both distally and proximally. Some embodiments of spring carrier 10 may comprise an end wall 17 which may assist with retaining a coil spring within the inner cavity. However, it will be appreciated that the end wall 17 may be omitted and the projecting element(s) 25A alone may be sufficient to retain the coil spring within the inner cavity 13. During a coil spring insertion process, an opening at the second, distal end may optionally be temporarily covered by some means, such as part of an assembly apparatus (not shown) until the projecting element(s) 25A engage and retain the coil spring within the inner cavity.

The embodiments of spring carrier 10 shown and described comprise resilient arms 18 having an actuation feature which is disposed at a distal-most end of the spring carrier. However, the disclosure is not intended to be limited to such a configuration, and in alternative embodiments, an actuation feature, such as a lip and contact surface, may be disposed spaced from a distal most end of the spring carrier. Such actuation feature may be less distal relative to an axial direction of the hollow body 11 than, for example, the flange 28. Such an alternative embodiment of spring carrier may function by the actuation feature engaging with an upstanding boss or other formation on the planar surface 31 to move the resilient arms 18 from the first position to the second position, when the flange 28 is in abutment with the planar surface. Furthermore, it is envisaged within the scope of the present disclosure that other means may be employed in an exemplary insertion/extraction process to effect movement of the resilient arms other than the exemplary embodiment of actuator actuation feature(s) that are shown and described. Some other external operator or mechanism (not shown) may engage and move the deflectable member(s) as required. Such an embodiment may again not require engagement with a specific actuation feature, or use of the specific actuator 30 shown and described, although these are not precluded within the scope of the present disclosure.

As mentioned above, throughout the present disclosure, it will be appreciated that the terms “inwardly” and “outwardly” are used as being generally relative to the body of the spring carrier 10. For example, relative to the central axis X-X or with respect to the hollow body 11/inner cavity 13 of the spring carrier 10. As such, as used herein, the deflectable member and/or retaining formation(s) being disposed or extending “outwardly” in a second deflected position will be understood as being disposed in a direction further away from the inner cavity 13 and/or axis X-X than when in a more inwardly-disposed position in a first, unbiased position. In some embodiments, as described above, the retaining formation(s) may be disposed outwardly of an inner surface of the inner cavity 13 in a second, biased position. This may help ensure a coil spring is discharged from the inner cavity 13. However, it will be appreciated that in alternative embodiments intended within the scope of the disclosure, the retaining formation(s) may be disposed further outwardly in the second, biased position than in a first, unbiased position, but not disposed outwardly of an inner surface of the inner cavity 13. It may be sufficient for the retaining formation(s) to be disposed further outwardly in the second, biased position such that a gap is provided which is at least larger than a coil spring diameter to allow the coil spring to pass out of the inner cavity 13. In an exemplary embodiment in which a coil spring has a diameter which is significantly smaller than an inner diameter of the inner cavity (but large enough to be retained by the retaining formation(s) when the deflectable member(s) is in the first, unbiased position), the retaining formation(s) may not need to be deflected outwardly of an inner surface of the inner cavity 13 in the second, biased position to disengage the coil spring to allow its release.

As will be appreciated from the various embodiments of spring carrier 10 shown and described above, the second end 15 of the hollow body 11 may be closed or may include an opening. Those embodiments of spring carrier 10 which comprise an opening 38 at the second end 15 of the hollow body 11 may have the opening which is of the same size and dimensions as the cross-sectional dimension of the inner cavity 13 of the hollow body 11, or an opening which is of a smaller cross-sectional dimension, and/or a different shape, than the cross-section of the inner cavity 13. Furthermore, those embodiments that comprise an opening 38 at the second end 15 of the hollow body 11, may comprise one or more protrusions extending inwardly to act as spring stops to retain a coil spring C in inner cavity 13 and prevent a coil spring C passing out of second end 15. Such protrusion(s) may be provided distal-most at the second end 15 of the hollow body 11, or may be spaced from the distal-most part of the second end 15 of the hollow body 11. However, the disclosure is not intended to be limited to configurations having an end wall or other protrusions acting as a spring stop, and in other embodiments within the scope of the disclosure, the second end 15 may include a second opening 38 of the same size and cross-sectional area and/or dimension as the cross-section of the inner cavity 13. Other means or methods may be provided in the apparatus with which the spring carrier 10 is to be used to prevent a coil spring C unintentionally passing out of the second end of the hollow body 11.

The embodiments of spring carrier 10 shown and described comprise projecting regions 25 proximate to the free ends 22 of the resilient arms 18. However, the disclosure is not intended to be limited to such a configuration and in alternative embodiments, one or more projecting regions may be disposed elsewhere on the resilient arms 18 intermediate the fixed end 21 and the free end 22.

The embodiments of actuation plate 30 shown and described comprise a feed aperture 32 comprising a tapered second region 34 and a first region 33 of generally constant cross-section. However, the disclosure is not intended to be limited to such a configuration and in alternative embodiments, the feed aperture 32 may be of constant cross-section along its entire length, or in another alternative embodiment, the feed aperture 32 may taper along its entire length. Furthermore, an apparatus of the disclosure including an actuation plate may comprise a different configuration of actuation plate for use during insertion of a coil spring C into the spring carrier 10 to the configuration of actuation plate for use during extraction of a coil spring C from the spring carrier 10. For example, the actuation plate for use during insertion of a coil spring C may not include a feed aperture 32 having a tapered region, whereas the actuation plate for use during extraction of a coil spring C may include a feed aperture 32 having a tapered region 34.

In any of the embodiments intended within the scope of the disclosure, the deflectable members may be spaced apart in the second position by a distance substantially equal to the diameter of the feed aperture 32 at the planar surface 31 of the actuation plate 30. In embodiments in which the feed aperture 32 includes a tapered region 34, the deflectable members may be spaced apart in the second position by a distance substantially equal to the diameter of the widest part of the tapered region 34.

Embodiments of spring carrier 10 described herein comprise at least one deflectable member configured to retain a coil spring within the inner cavity 13 of the hollow body 11. The at least one deflectable member is provided proximate to an end of the hollow body 11 into and from which a coil spring C is inserted/extracted in use. This arrangement may help achieve blocking and releasing function of the projecting regions 25 that retain/allow release of a coil spring C within/from the inner cavity 13. This may help towards providing a simple and reliable manufacturing/assembly apparatus and process. Furthermore, in the exemplary embodiments illustrated and described, the engagement of the deflectable member(s) and/or retaining portion(s) and/or projecting element(s) with the coil spring to retain the coil spring, is effected by direct contact between the deflectable member(s) and/or retaining formation(s) and/or projecting element(s) with the coil spring.

Some embodiments disclosed herein comprise a flange 28 extending around the perimeter of the hollow body 11 at the first proximal end 14 thereof. Such feature may optionally be applicable to all embodiments described herein. However, the disclosure is not intended to be limited to such feature and embodiments envisaged within the scope of the disclosure may not comprise a flange 28, or may comprise a flange disposed along the length of the hollow body other than at the remote end of the first proximal end, for example, at the second distal end 15, or intermediate the first proximal end and the second distal end.

The embodiments of spring carrier 10 shown and described comprise a lip 23 at the free end of each deflectable member/resilient arm 18 as the actuation feature, for engagement to move the resilient arms from the first position to the second position. However, the disclosure is not intended to be limited to such a configuration and in alternative embodiments, a lip 23 may be omitted from each resilient arm 18, and a free end 22 of each arm may instead just be configured with an end to engage with an actuation plate to be moved into the second position. In such an embodiment, the second position of the resilient arms 18 may be reached at the point the flange 28 abuts the planar surface 31 of the actuation plate 30. As discussed above, the resilient arms may not include a specific actuation feature and may otherwise be manipulated in use to deflect and move as needed. For example, such alternative external actuator may effect mechanical engagement with the arm(s), such as by adhesion, vacuum contact, or other coupling.

Those of skill in the art will understand that modifications (additions and/or removals) of various components of the devices, apparatuses, methods, and embodiments described herein may be made without departing from the full scope and spirit of the present disclosure, which encompass such modifications and any and all equivalents thereof.

1-17. (canceled)

18. A spring carrier for receiving, retaining, and discharging of a coil spring in a manufacturing assembly process, the spring carrier comprising:
an elongate hollow body defining an inner cavity configured to receive the coil spring;
an opening at a first proximal end of the hollow body for insertion of the coil spring into the inner cavity and extraction of the coil spring from the inner cavity;
the hollow body including a second distal end opposite to the first proximal end; and
at least one deflectable member located proximate to the first proximal end of the hollow body and including a retaining portion configured to retain the coil spring when the coil spring is located within the inner cavity,
wherein the at least one deflectable member is movable between a first unbiased position, whereby the retaining portion extends into the inner cavity to retain the coil spring within the inner cavity, and a second biased position, whereby the retaining portion is disposed outwardly to allow the coil spring to be extracted from the inner cavity through the opening.

19. The spring carrier according to claim 18, wherein the at least one deflectable member extends substantially parallel to a central axis of the hollow body in the first unbiased position.

20. The spring carrier according to claim 18, wherein the at least one deflectable member includes an actuation feature for engagement with an actuator to deflect the at least one deflectable member from the first unbiased position to the second biased position.

21. The spring carrier according to claim 20, wherein the actuation feature comprises a contact surface disposed at an acute angle with respect to a central axis of the hollow body when the at least one deflectable member is in the first unbiased position.

22. The spring carrier according to claim 21, wherein the contact surface is disposed further in a proximal direction than a remainder of the hollow body when the at least one deflectable member is in the first unbiased position.

23. The spring carrier according to claim 22, wherein the contact surface is disposed substantially perpendicular with respect to the central axis of the hollow body when the at least one deflectable member is in the second biased position.

24. The spring carrier according to claim 21, wherein the contact surface is disposed substantially perpendicular with respect to the central axis of the hollow body when the at least one deflectable member is in the second biased position.

25. The spring carrier according to claim 21, wherein the hollow body includes a flange proximate the first proximal end of the hollow body and extending radially outwardly from the hollow body.

26. The spring carrier according to claim 25, wherein the contact surface extends in the same plane as an abutment surface of the flange when the at least one deflectable member is in the second biased position.

27. The spring carrier according to claim 18, wherein the retaining portion comprises at least one projecting region extending inwardly from the at least one deflectable member.

28. The spring carrier according to claim 18, wherein the hollow body includes a flange proximate the first proximal end of the hollow body and extending radially outwardly from the hollow body.

29. The spring carrier according to claim 18, wherein the hollow body comprises one or more protrusions extending inwardly at least partially across a second opening at the second distal end of the hollow body.

30. The spring carrier according to claim 18, comprising at least one window in at least one of a side wall of the hollow body or the at least one deflectable member to allow the coil spring located within the spring carrier to be visible from outside the spring carrier through the at least one window.

31. An apparatus comprising:
a spring carrier comprising:
an elongate hollow body defining an inner cavity configured to receive a coil spring;
an opening at a first proximal end of the hollow body for insertion of the coil spring into the inner cavity and extraction of the coil spring from the inner cavity;
the hollow body including a second distal end opposite to the first proximal end; and
at least one deflectable member located proximate to the first proximal end of the hollow body and including a retaining portion configured to retain the coil spring when the coil spring is located within the inner cavity;
wherein the at least one deflectable member is movable between a first unbiased position, whereby the retaining portion extends into the inner cavity to retain the coil spring within the inner cavity, and a second biased position, whereby the retaining portion is disposed outwardly to allow the coil spring to be extracted from the inner cavity through the opening; and
an actuator configured for engagement with the at least one deflectable member and operable to move the at least one deflectable member from the first unbiased position to the second biased position.

32. The apparatus according to claim 31, wherein the actuator comprises an actuation plate and includes a feed aperture therethrough for alignment with the inner cavity of the hollow body such that the coil spring can be inserted into the inner cavity through the feed aperture and extracted from the inner cavity through the feed aperture when the spring carrier is engaged with the actuator.

33. A method of manipulating a coil spring using a spring carrier for receiving, retaining, and discharging of the coil spring in a manufacturing assembly process, the spring carrier comprising an elongate hollow body defining an inner cavity, an opening at a proximal first end of the hollow body, a second distal end opposite to the first proximal end, and at least one deflectable member located proximate to the first proximal end of the hollow body and including a retaining portion, the method comprising:
moving the at least one deflectable member from a first position whereby the retaining portion extends into the inner cavity, to a second position whereby the retaining portion is disposed outwardly,
inserting the coil spring into the inner cavity through the opening at the first proximal end of the hollow body, and
moving the at least one deflectable member from the second position to the first position such that the retaining portion extends into the inner cavity to retain the coil spring within the inner cavity.

34. The method of claim 33, wherein the method comprises:
engaging an actuator with the at least one deflectable member to move the at least one deflectable member from the first position to the second position, and
disengaging the actuator after insertion of the coil spring into the inner cavity to allow the at least one deflectable member to move to the first position such that the retaining portion retains the coil spring within the inner cavity.

35. The method of claim 34, wherein engaging the actuator with the at least one deflectable member comprises pressing the first proximal end of the spring carrier against a planar surface of the actuator such that the at least one deflectable member contacts the planar surface and the at least one deflectable member is moved to the second position.

36. The method of claim 35, wherein the actuator comprises an actuation plate, and inserting the coil spring into the inner cavity comprises passing the coil spring through a feed aperture in the actuation plate and into the inner cavity.

37. The method of claim 34, wherein the actuator comprises an actuation plate, and inserting the coil spring into the inner cavity comprises passing the coil spring through a feed aperture in the actuation plate and into the inner cavity.
